(19) United States
(12) Patent Application Publication
(10) Pub. No.: US 2025/0232832 A1
(43) Pub. Date: Jul. 17, 2025

(54) METHOD, DEVICE AND MEDIUM FOR PROTEIN LANGUAGE MODEL

(71) Applicants: Bytedance Technology Ltd., Grand Cayman (KY); Beijing Youzhuju Network Technology Co., Ltd., Pinggu District, Beijing (CN)

(72) Inventors: Cheng-Yen Hsieh, Los Angeles, CA (US); Xinyou Wang, Beijing (CN); Dongyu Xue, Beijing (CN); Fei Ye, Beijing (CN); Zaixiang Zheng, Beijing (CN); Quanquan Gu, Los Angeles, CA (US)

(21) Appl. No.: 19/097,452

(22) Filed: Apr. 1, 2025

(51) Int. Cl. G16B 15/20; G16B 40/20

(52) U.S. Cl. CPC G16B 15/20; G16B 40/20

(57) ABSTRACT

Embodiments of the disclosure provide a solution for a protein language model. A method includes: obtaining a sequence representation of a protein comprising a plurality of amino acid residues, the sequence representation characterizing an amino acid sequence of the protein; determining, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue; and generating a target structure of the protein based on the predicted discrete structure representation.

### FIELD

The present disclosure generally relates to computer technologies, and more specifically, to a method, apparatus, device and computer readable storage medium for a protein language model.

### BACKGROUND

Proteins are the molecular machinery of life, encoded by amino acid sequences that fold into intricate three-dimensional structures to perform their biological functions. Conventional approaches often treat a sequence and a structure of the protein as separate modalities, relying on disjoint models that fail to capture the interplay between them. This limitation hinders the ability to jointly model, understand, and generate proteins in a unified framework, which is essential for tasks like protein design, folding, and functional annotation.

### SUMMARY

In a first aspect of the present disclosure, there is provided a method of protein structure generation. The method comprises: obtaining a sequence representation of a protein comprising a plurality of amino acid residues, the sequence representation characterizing an amino acid sequence of the protein; determining, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue; and generating a target structure of the protein based on the predicted discrete structure representation.

In a second aspect of the present disclosure, there is provided a method of protein structure generation. The method comprises: applying, by a language model, a first attention mechanism to a structure representation of a protein and a sequence representation of the protein, to obtain a first updated structure representation of the protein and a first updated sequence representation of the protein; applying, by the language model, a second attention mechanism to a pair representation of the protein and the first updated structure representation, to obtain an updated pair representation of the protein and a second updated structure representation of the protein, wherein the pair representation characterizes interactions between pairs of amino acid residues in the protein; applying, by the language model, a third attention mechanism to the updated pair representation, the second updated structure representation and the first updated sequence representation, to obtain at least one of a predicted structure representation of the protein or a predicted sequence representation of the protein.

In a third aspect of the present disclosure, there is provided an apparatus for protein structure generation. The apparatus comprises: an obtaining module configured to obtain a sequence representation of a protein comprising a plurality of amino acid residues, the sequence representation characterizing an amino acid sequence of the protein; a determining module configured to determine, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue; and a generating module configured to generate a target structure of the protein based on the predicted discrete structure representation.

In a fourth aspect of the present disclosure, there is provided an apparatus for protein structure generation. The apparatus comprises: a first applying module configured to apply, by a language model, a first attention mechanism to a structure representation of a protein and a sequence representation of the protein, to obtain a first updated structure representation of the protein and a first updated sequence representation of the protein; a second applying module configured to apply, by the language model, a second attention mechanism to a pair representation of the protein and the first updated structure representation, to obtain an updated pair representation of the protein and a second updated structure representation of the protein, wherein the pair representation characterizes interactions between pairs of amino acid residues in the protein; and a third applying module configured to apply, by the language model, a third attention mechanism to the updated pair representation, the second updated structure representation and the first updated sequence representation, to obtain at least one of a predicted structure representation of the protein or a predicted sequence representation of the protein.

In a fifth aspect of the present disclosure, there is provided an electronic device. The electronic device comprises: at least one processor; and at least one memory coupled to the at least one processor and storing instructions executable by the at least one processor, the instructions, upon execution by the at least one processor, causing the electronic device to perform: obtaining a sequence representation of a protein comprising a plurality of amino acid residues, the sequence representation characterizing an amino acid sequence of the protein; determining, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue; and generating a target structure of the protein based on the predicted discrete structure representation.

In a sixth aspect of the present disclosure, there is provided an electronic device. The electronic device comprises: at least one processor; and at least one memory coupled to the at least one processor and storing instructions executable by the at least one processor, the instructions, upon execution by the at least one processor, causing the electronic device to perform: applying, by a language model, a first attention mechanism to a structure representation of a protein and a sequence representation of the protein, to obtain a first updated structure representation of the protein and a first updated sequence representation of the protein; applying, by the language model, a second attention mechanism to a pair representation of the protein and the first updated structure representation, to obtain an updated pair representation of the protein and a second updated structure representation of the protein, wherein the pair representation characterizes interactions between pairs of amino acid residues in the protein; applying, by the language model, a third attention mechanism to the updated pair representation, the second updated structure representation and the first updated sequence representation, to obtain at least one of a predicted structure representation of the protein or a predicted sequence representation of the protein.

In a seventh aspect of the present disclosure, a computer-readable storage medium is provided. The computer-readable storage medium stores computer executable instructions which, when executed by an electronic device, causes the electronic device perform operations comprising: obtaining a sequence representation of a protein comprising a plurality of amino acid residues, the sequence representation characterizing an amino acid sequence of the protein; determining, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue; and generating a target structure of the protein based on the predicted discrete structure representation.

In an eighty aspect of the present disclosure, a computer-readable storage medium is provided. The computer-readable storage medium stores computer executable instructions which, when executed by an electronic device, causes the electronic device perform operations comprising: applying, by a language model, a first attention mechanism to a structure representation of a protein and a sequence representation of the protein, to obtain a first updated structure representation of the protein and a first updated sequence representation of the protein; applying, by the language model, a second attention mechanism to a pair representation of the protein and the first updated structure representation, to obtain an updated pair representation of the protein and a second updated structure representation of the protein, wherein the pair representation characterizes interactions between pairs of amino acid residues in the protein; applying, by the language model, a third attention mechanism to the updated pair representation, the second updated structure representation and the first updated sequence representation, to obtain at least one of a predicted structure representation of the protein or a predicted sequence representation of the protein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE SEVERAL VIEWS OF THE DRAWINGS

The above and other features, advantages and aspects of the embodiments of the present disclosure will become more apparent in combination with the accompanying drawings and with reference to the following detailed description. In the drawings, the same or similar reference symbols refer to the same or similar elements, where:

FIG. 1 illustrates a schematic diagram of an example environment in which embodiments of the present disclosure may be implemented;

FIG. 2A illustrates a schematic diagram of a process of restructuring a protein structure;

FIG. 2B illustrates a schematic diagram of a process of training a language model;

FIG. 3A illustrates a schematic diagram of an example process of generating a target structure of a protein in accordance with some embodiments of the present disclosure;

FIG. 3B illustrates a schematic diagram of an example process of recovering a residual in accordance with some embodiments of the present disclosure;

FIG. 3C illustrates a schematic diagram of an example process of sampling with a flow-based sampler in accordance with some embodiments of the present disclosure;

FIG. 4 illustrates a schematic diagram of a structure of the language model in accordance with some embodiments of the present disclosure;

FIG. 5 illustrates a schematic diagram of a structure of a structure attention module in accordance with some embodiments of the present disclosure;

FIG. 6 illustrates a schematic diagram of a structure of a sequence structure attention module in accordance with some embodiments of the present disclosure;

FIG. 7 illustrates a schematic diagram of a process of aligning the representations of the language model to the representations of a folding model;

FIG. 8 illustrates a flowchart of a method of protein structure generation in accordance with some example implementations of the present disclosure;

FIG. 9 illustrates a flowchart of another method of protein structure generation in accordance with some example implementations of the present disclosure;

FIG. 10 shows a block diagram of an apparatus for protein structure generation in accordance with some embodiments of the present disclosure;

FIG. 11 shows a block diagram of another apparatus for protein structure generation in accordance with some embodiments of the present disclosure; and

FIG. 12 illustrates a block diagram of an electronic device in which one or more embodiments of the present disclosure can be implemented.

### DETAILED DESCRIPTION

The embodiments of the present disclosure will be described in more detail below with reference to the accompanying drawings. Although some embodiments of the present disclosure are shown in the drawings, it would be appreciated that the present disclosure may be implemented in various forms and should not be interpreted as limited to the embodiments described herein. On the contrary, these embodiments are provided for a more thorough and complete understanding of the present disclosure. It would be appreciated that the drawings and embodiments of the present disclosure are only for the purpose of illustration and are not intended to limit the scope of protection of the present disclosure.

In the description of the embodiments of the present disclosure, the term “including” and similar terms would be appreciated as open inclusion, that is, “including but not limited to”. The term “based on” would be appreciated as “at least partially based on”. The term “one embodiment” or “the embodiment” would be appreciated as “at least one embodiment”. The term “some embodiments” would be appreciated as “at least some embodiments”. Other explicit and implicit definitions may also be included below. As used herein, the term “model” can represent the matching degree between various data. For example, the above matching degree can be obtained based on various technical solutions currently available and/or to be developed in the future.

It will be appreciated that the data involved in this technical proposal (including but not limited to the data itself, data acquisition or use) shall comply with the requirements of corresponding laws, regulations and relevant provisions.

It will be appreciated that before using the technical solution disclosed in each embodiment of the present disclosure, users should be informed of the type, the scope of use, the use scenario, etc. of the personal information involved in the present disclosure in an appropriate manner in accordance with relevant laws and regulations, and the user's authorization should be obtained.

For example, in response to receiving an active request from a user, a prompt message is sent to the user to explicitly prompt the user that the operation requested operation by the user will need to obtain and use the user's personal information. Thus, users may select whether to provide personal information to the software or the hardware such as an electronic device, an application, a server or a storage medium that perform the operation of the technical solution of the present disclosure according to the prompt information.

As an optional but non-restrictive implementation, in response to receiving the user's active request, the method of sending prompt information to the user may be, for example, a pop-up window in which prompt information may be presented in text. In addition, pop-up windows may also contain selection controls for users to choose “agree” or “disagree” to provide personal information to electronic devices.

It will be appreciated that the above notification and acquisition of user authorization process are only schematic and do not limit the implementations of the present disclosure. Other methods that meet relevant laws and regulations may also be applied to the implementation of the present disclosure.

As used herein, the term “model” is referred to as an association between an input and an output learned from training data, and thus a corresponding output may be generated for a given input after the training. The generation of the model may be based on a machine learning technique. In general, a machine learning model may be built, which receives input information and makes predictions based on the input information. For example, a classification model may predict a class of input information among a predetermined set of classes. As used herein, “model” may also be referred to as “machine learning model”, “learning model”, “machine learning network”, or “learning network,” which are used interchangeably herein.

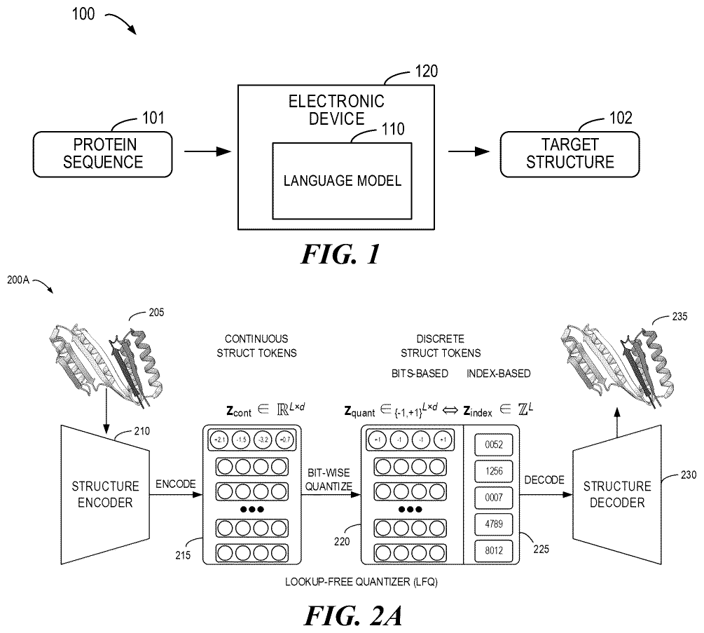

FIG. 1 illustrates a block diagram of an example environment 100 in which various embodiments of the present disclosure may be implemented. In the environment 100 of FIG. 1, a language model 110 is deployed in an electronic device 120. The electronic device 120 receives in formation of a protein sequence (e.g., chemical composition sequence) 101 of a protein. Then, the electronic device 120 applies the language model 110 to generate a target structure of the protein based on the protein sequence 101.

In the environment 100 of FIG. 1, the electronic device 120 may include any computing system with computing capability, such as various computing devices/systems, terminal devices, servers, etc. Terminal devices may include any type of mobile terminals, fixed terminals, or portable terminals, including mobile phones, desktop computers, laptops, netbooks, tablets, media computers, multimedia tablets, or any combination of the aforementioned, including accessories and peripherals of these devices or any combination thereof. Servers include but are not limited to mainframe, edge computing nodes, computing devices in cloud environment, etc.

As mentioned above, conventional approaches often treat the sequence and the structure as separate modalities. Recent works in multimodal protein language models have demonstrated the potential of integrating the sequence and the structure within a single language model as a unified generative framework. In an example, a multimodal extension of a diffusion protein language model with discrete diffusion framework aligns with the discrete nature of protein sequences, enabling it to benefit from large-scale pre-training on sequence databases which is a factor for accurate structure prediction. Beyond sequence modeling, this multimodal protein language model extends its capabilities by tokenizing three-dimensional (3D) coordinates into discrete tokens, thereby enabling direct modeling, comprehension and generation of both modalities. In addition, a structure tokenization process introduces structural information loss which obscures fine-grained geometric relationships critical for accurate protein modeling. However, this multimodal protein language model (PLM) struggles to generate biologically plausible structures for complex tasks like structure folding or motif scaffolding, where precise structural correlations are crucial. The loss of nuanced variations due to tokenization also degrades the structure diversity in unconditional generation.

The aim of generative protein modeling is to estimate the underlying distribution prot˜q(prot) of all associated modalities of the protein by learning a probabilistic model p_{θ}(prot). Here prot=(r₁, r₂, . . . , r_{L}) denotes a protein with L residues, where each residue rᵢ=(sᵢ, xᵢ) is represented by two major modalities, that is, sᵢ∈{0,1}^{|S|} is a categorical variable for its amino acid type in S={1, . . . ,20} and xᵢ∈ [symbol] ^{N}ₐₜₒₘₛ^{×3} represents the real-value Cartesian coordinates of its residue atoms (only backbone atoms are considered herein, that is, [N, C_{α}, C, O] with Nₐₜₒₘₛ=4). That is, p_{θ}(s, x)=p_{θ}(s₁, s₂, . . . , s_{L}, x₁, x₂, . . . , x_{L}).

Multimodal generative approaches that jointly model the structure and the sequence may be mainly categorized into two paradigms, that is, structure-centered diffusion/flow-based models or sequence-centered language models. The latter is mainly illustrated in the present disclosure as an example without limitation.

Language models (LMs) parameterized by large-scale Transformers have become a choice dominating different domains with scalable and performing expressiveness. Among them, protein LMs have been serving as one of the AI foundations for protein sequence learning and generation.

A diffusion protein language model (DPLM) shows excelling performance in both generation and representation learning of protein sequences and structures. The DPLM grounded in absorbing discrete diffusion framework, which is characterized by a forward and backward Markov process. Let Cat(x; p) be a categorical distribution on a protein sequence y parameterized by a vector p on (|V|−1)-dimensional probability simplex. The forward process of discrete diffusion defines a Markov process governed by the transition kernel q(x^{(t)}|x^{(t-1)})=Cat(x^{(t)};βₜx^{(t-1)}+(1−βₜ)qₙₒᵢₛₑ) that gradually perturb the data x˜q(x) into a stationary distribution x^{(T)}˜qₙₒᵢₛₑ. The learned backward process p_{θ}(x^{(t-1)}|x^{(t)}) reversely denoises the x^{(T)} towards the data distribution x, which is typically optimized by the variational bound of the log-likelihood. The training objective of absorbing diffusion is as follows, may be simplified into weighted cross-entropies, resembling masked language modeling at arbitrary noise levels:

$$\begin{matrix}{𝒥}_{t} = {𝔼}_{q ( x )} - K L [ q ( {x}^{( t - 1 )} \vert {x}^{( t )} , x ) ‖ {p}_{θ} ( {x}^{( t - 1 )} \vert {x}^{( t )} ) ] \\ = {𝔼}_{q ( x )} [ {λ}^{( t )} {\sum }_{1 \leq i \leq L} {b}_{i} ( t ) \cdot log {p}_{θ} ( {x}_{i} \vert {x}^{( t )} ) ] ,\end{matrix} \qquad ( 1 )$$

where λ^{(t)} represents a weighting coefficient induced from a specific noising schedule, t represents a time step, KL [symbol] represents a Kullback-Leibler divergence, x represents a structure of a protein and bᵢ(t) represents a probability of the i-th action at the time step t. For inference, the DPLM may generate amino acid sequences by a reverse iterative denoising process in a mask-predict manner, which starts from an all-mask sequence and iterates towards a synthesized sequence. At time t, the DPLM first generates \tilde{X}(0) from p_{θ}(·|x^{(t)}), then a less noisy x^{(t-1)} is sampled by q(·|x^{(t)}, x⁽⁰⁾=\tilde{x}⁽⁰⁾).

To facilitate structure learning in language models, the DPLM may be extended by introducing a token-based latent representation for a protein structure and this is achieved via a two-stage approach. Firstly, a structure tokenizer learns to convert x∈ [symbol] ^{L×Nbackb×3}, the 3D coordinates of the protein backbone into a discrete structure token sequence, denoted as z=(z₁, z₂, . . . , z_{L})∈{0 . . . | [symbol] |}^{L}, where each token zᵢ represents a local structural element of the i-th residue and | [symbol] | is the codebook size. Secondly, given a tokenized structure, the DPLM processes multimodal input, and then performs joint language modeling of the structure token sequence z with the corresponding amino acid sequence s for the same protein. The training objective of the DPLM hence becomes:

$${𝒥}_{t} = {𝔼}_{q ( x , z )} [ {λ}^{( t )} {\sum }_{i} {b}_{i} ( t ) ( log {p}_{θ} ( {s}_{i} \vert \cdot ) + log {p}_{θ} ( {z}_{i ,} \vert \cdot ) ) ] . \qquad ( 2 )$$

Any vector-quantization based approach may be studied for tokenizing protein atomic structure into structure tokens. The DPLM employs a lookup-free quantization (LFQ)-based structure tokenizer, which is used for visual tokenization. This LFQ tokenizer may be summarized as follows:

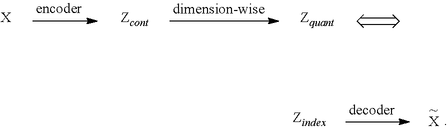

At a first step, a structure encoder encodes backbone 3D coordinates x∈ [symbol] ^{L×Nbackb×3} into invariant features as continuous structure tokens z_{cont}∈ [symbol] ^{L×D}. At a second step, an LFQ module quantizes z_{cont} independently dimension-wise into bits-based (binary) discrete structure tokens z_{quant}∈{−1, +1}^{L×D}, which can be converted to decimal index-based discrete structure tokens Z_{index}=Σₖ^{D}1(z_{quant}^{[k]}>0)·2^{k-1}∈{0 . . . |Z|}^{L}. At a third step, a structure decoder reconstructs 3D coordinates from the discrete tokens.

Although discrete structure tokens are important to multimodal protein language models, using discrete structure tokens to represent structural information also limits the ability of the model to capture structural details accurately. This trade-off represents a challenge in the current field of multimodal protein language models and an in-depth study regarding structure tokenization and structure prediction by LMs may be conducted. In addition, some observations have been obtained.

A first observation indicates that structure tokenization results in information loss. Vector quantization converts latent features of continuous structure tokens (z_{cont}) into discrete structure tokens (z_{quant}), discarding residual information (z_{cont}−z_{quant}). Therefore, applying quantization amplifies reconstruction errors. This indicates that quantizing continuous tokens into discrete tokens inevitably results in loss of fidelity and detailed structural accuracy. This suggests that learning to recover the lost residuals, e.g., as a refinement step, may enhance structure prediction accuracy.

A second observation indicates that high reconstruction accuracy does not guarantee better structure generative performance in language models, while a significant gap remains in between. The impact of different protein structure tokenizers on reconstruction and generation tasks are compared in the study. Two tokenizers are selected to train separate DPLM variants with the same architecture but using their respective structure token codebook. These models are evaluated for both reconstruction and protein folding (e.g., generation) performance. Based on the comparing result, a first tokenizer achieves superior reconstruction accuracy outperforming a second tokenizer. However, the DPLM trained with the codebook of the second tokenizer exhibits stronger protein folding performance. This indicates that while reconstruction accuracy sets an upper bound on generation quality, the substantial gap between reconstruction and generation highlights the critical role of the LMs' generative capability in structure prediction. In addition, this suggests that mild improvement in reconstruction do not necessarily translate into better generation, greater emphasis should be placed on improving structure-aware generative modeling and architectural design.

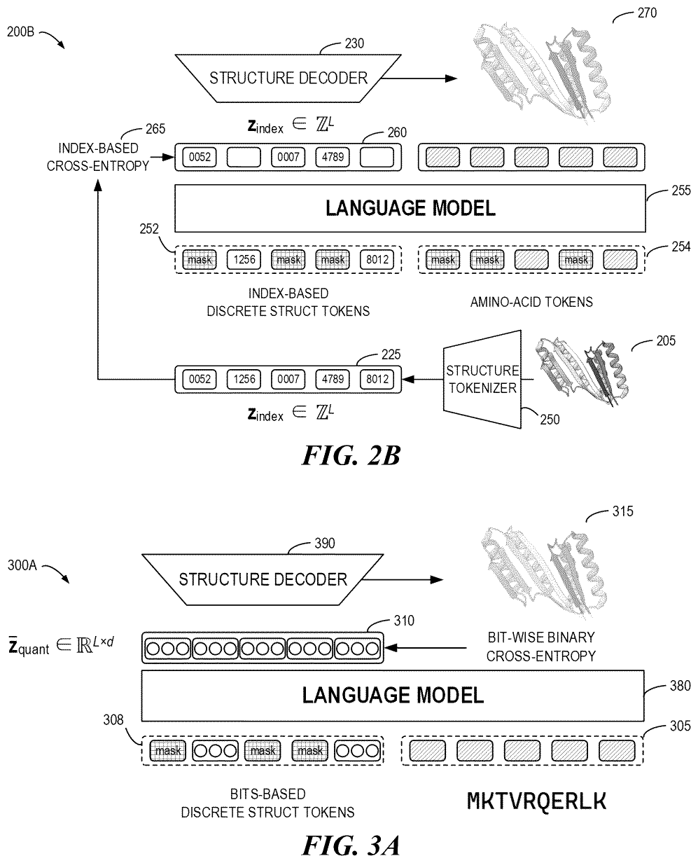

In order to better illustrate a third observation of the present disclosure, a structure tokenizer is now described with reference to FIG. 2A and FIG. 2B. FIG. 2A illustrates a schematic diagram of a process 200A of restructuring a protein structure. As shown in FIG. 2A, a structure encoder 210 may encode a protein structure 205 into continuous structure tokens 215. A continuous structure token may be represented by positive or negative numbers. The continuous structure tokens 215 may be quantized, by the structure tokenizer (not shown), into bits-based structure tokens 220 (as an example of discrete structure tokens). For example, the positive number may be quantized into +1 and the negative number may be quantized into −1. The bits-based structure tokens 220 may be converted, by the structure tokenizer, into index-based structure tokens 225 (as another example of discrete structure tokens). Then, a structure decoder 230 may decode the index-based structure tokens 225 into a reconstructed protein structure 235.

FIG. 2B illustrates a schematic diagram of a process 200B of training a language model. As shown in FIG. 2B, a structure tokenizer 250 may quantize the protein structure 205 into the index-based structure tokens 225. At least one token in the index-based structure tokens 225 is masked and at least one token in sequence tokens (e.g., amino-acid tokens) of the protein 205 may be masked. Then, a language model 255 (as an implementation of the language model 110) may generate predicted index-based structure tokens 260 based on the masked index-based structure tokens 252 and the masked sequence tokens 254. The structure decoder 230 may decode the predicted index-based structure tokens 260 to obtain a predicted protein structure 270. In addition, a loss 265 (e.g., an index-based cross-entropy) between the index-based structure tokens 225 and the predicted index-based structure tokens 260 may be determined. The language model 255 may be trained based on an objective, which is configured to reduce or minimize the loss 265.

The third observation indicates that direct index prediction (e.g., prediction based on the index-based structure tokens) is inaccurate. However, the structural evaluation metrics, such as root-mean-square deviation (RMSD) and/or TMscore, indicate that the generated structures do not completely collapse, suggesting that despite the coarse-grained supervision, the model still captures some underlying relationships between indices. This learning process, however, remains highly challenging: since each index is derived from multiple quantized bits, even small changes at the bit level can result in drastically different indices. This issue becomes even more problematic as the codebook size increases, further exacerbating the difficulty of direct index prediction. In contrast, when evaluated at the bit-based level, prediction accuracy aligns more closely with structural evaluation metrics. This suggests that while the model struggles to recover exact indices, it effectively captures structural patterns at the bit level.

In order to solve at least some of the above technical problems, embodiments of the present disclosure propose an improved solution for PLMs. In this solution, a sequence representation of a protein comprising a plurality of amino acid residues is obtained. A predicted discrete structure representation is determined, by a language model, based on the sequence representation. The predicted discrete structure representation comprises a plurality of bit sequences corresponding to the plurality of amino acid residues respectively. A bit sequence of the plurality of bit sequences represents a predicted local structure of a corresponding amino acid residue. A target structure of the protein is generated based on the predicted discrete structure representation.

With these embodiments of the present disclosure, the bit-based predicted discrete structure representation is determined which may effectively capture more structural patterns of the protein and provide finer-grained supervision. In this way, the structure prediction accuracy of the language model may be improved while the structural deviation from ground truth may be reduced.

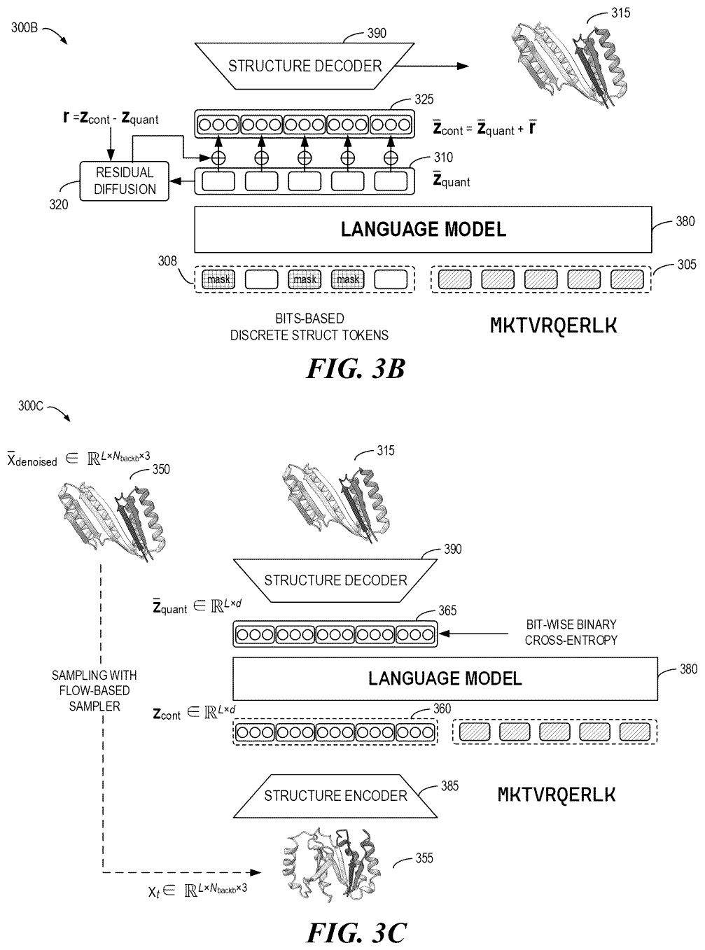

Discretizing protein structures into index-based tokens enables multimodal PLMs to perform structural modeling but introduces challenges, as discussed in the third observation. However, bit-based tokens may provide more informative supervision signals and thus embodiments of the present disclosure perform language modeling of the bit-based feature of structure tokens instead of their indices. The language modeling of the bit-based feature of structure tokens may be introduced with reference to FIG. 3A, which illustrates a schematic diagram of an example process 300A of generating a target structure of a protein in accordance with some embodiments of the present disclosure. As shown in FIG. 3A, a sequence representation 305 of a protein comprising a plurality of amino acid residues is obtained. The sequence representation characterizes an amino acid sequence of the protein. In some examples, the sequence representation 305 may be obtained by encoding the amino acid sequence using a sequence encoder.

After obtaining sequence representation 305, a predicted discrete structure representation 310 is determined by the language model 380 (for example, a DPLM). The predicted discrete structure representation 310 comprises a plurality of bit sequences corresponding to the plurality of amino acid residues respectively. A bit sequence represents a predicted local structure of a corresponding amino acid residue. In the example of FIG. 3A, the bit sequence includes three bits. However, this is an example without any limitation. Such a bit sequence may be referred to as a bits-based discrete structure token. In some embodiments, to predict a discrete structure representation, an input structure representation of the protein may be provided to the language model 380 as another input. The input structure representation may include tokens corresponding to the plurality of amino acid residues. A token may be a bit sequence, or in other words, the bits-based discrete token.

In an inference process of the language model 380, all tokens in the input structure representation of the protein may be masked or include noise and thus the structure representation is unknown to the language model 380. The language model 380 may only rely on the sequence representation 305 to determine the predicted discrete structure representation 310. Then, a target structure 315 of the protein is generated based on the predicted discrete structure representation 310. In an example, the structure decoder 390 may decode the predicted discrete structure representation 310 into the target structure 315. In some embodiments, the language model 380 may be constructed based on a diffusion model.

The scarcity of protein structure data may be a concern for developing multimodal protein language models (e.g., the language model 380). Currently, multimodal protein language models are trained based on data including single-chain proteins (also referred to as monomer data). In some embodiments, the protein may comprise at least one of a multi-chain protein or a single-chain protein. The single-chain protein includes a single polypeptide chain, which indicates that it has only one continuous sequence of amino acids folded into a functional structure. The multi-chain protein includes two or more polypeptide chains, which allows for more complex functions and interactions. Data including multi-chain proteins (also referred to as multimer data) used to train the language model 380 presents diverse structural arrangements and interaction scenarios, which are essential for developing a more general multimodal protein language model. In this way, the structural modeling performance of the language model 380 may be improved.

In some examples, the gap between multimer and monomer data may be identified and bridged. It is to be noted that chains are typically spaced farther apart than individual connecting residues, a position index offset is applied to each residue, which is calculated as the product of chain index and a predefined offset value. The offset is incorporated into the relative position embedding of the structure detokenizer. The effects of connecting chains are examined using glycine (G) linkers of varying lengths under the folding scenario. These linkers not only introduce a position offset but also serve as pseudo-connectors between protein chains. Based on findings of the present disclosure, chain linkers and position offsets both improve metrics (e.g., RMSD or TMscore) of reconstruction performance of the structure tokenizer. These results highlight the difference between multimer and monomer and suggest that properly differentiating chains in sequence and positional space are essential for effective multimer modeling.

In some embodiments, as shown in FIG. 3A, in a training process of the language model 380, the predicted discrete structure representation 310 may be determined further based on a masked discrete structure representation 308 of the protein. In other words, in the training process, the input structure representation to the language model 380 is the masked discrete structure representation 308. The masked discrete structure representation 308 may be generated by masking at least one bit sequence in a sample discrete structure representation of the protein. A bit sequence in the sample discrete structure representation may correspond to an amino acid residue of the plurality of amino acid residues and represents a sample local structure of the corresponding amino acid residue.

A loss function (e.g., a bit-wise binary cross-entropy loss function) may be determined based on a difference between the predicted discrete structure representation 310 and the sample discrete structure representation. The sample discrete structure representation may be considered as a ground truth for the predicted discrete structure representation 310. Then, the language model 380 may be updated based on the loss function. In some examples, the language model 380 may be trained based on a training objective, which is configured to reduce or minimize a value of the loss function. In this way, more structural details of the protein may be reserved while remaining compatible with a discrete supervision of the PLM. It hence becomes K binary classifications to predict each bit of K-bit structure token, instead of the original 2^{K-1}-way classifications. This greatly reduces the training challenges and thus improves generative accuracy. As such, the training objective with bits-based structure modeling is accordingly modified as:

$${𝒥}_{t}^{b i t} = {𝔼}_{q ( x , z )} [ {λ}^{( t )} {\sum }_{i} {b}_{i} ( t ) ( log {p}_{θ} ( {s}_{i} \vert \cdot ) + {\sum }_{k} log {p}_{θ} ( {z}_{i , quant}^{[ k ]} \vert \cdot ) ) ] \qquad ( 3 )$$

In some experiments conducted according to embodiments of the present disclosure, it is observed that bit-level supervised language model achieves generative accuracy improvements across both index-level and bit-level, while substantially reduced structural deviation from ground truth. This suggests that the fine-grained bit-level supervision signals are more suitable for training the language model 380, enabling the language model to capture structural patterns more effectively, which enhances the latent structural modeling.

In the structure tokenizer, continuous structure is converted into discrete structure token features, fundamentally clustering similar local environments into identical token. However, according to the first observation, this process inherently introduces lossy compression, residuals, i.e., differences between the continuous features and the discrete features are lost during this process, eliminating fine-grained structural details. In order to further improve the generative accuracy of the language model 380, embodiments of the present disclosure recover and preserve the high-frequency variation that gets lost during tokenization process.

FIG. 3B illustrates a schematic diagram of an example process 300B of recovering a residual in accordance with some embodiments of the present disclosure. As shown in FIG. 3B, a residual (denoted as r̅) of the predicted discrete structure representation 310 relative to a continuous structure representation of the protein may be determined based on a hidden state of the language model 380 and the predicted discrete structure representation 310. In some examples, a residual diffusion module 320 (e.g., constructed based on a diffusion model) may predict the residual conditioned on the hidden states (denoted as h) of the language model 380 and the predicted discrete structure representation 310. In a training process, the ground truth residual for training the residual diffusion module 320 may be denoted as r=z_{cont}−z_{quant}, where z_{cont} and z_{quant} represents ground truth structure representations of the protein. Specifically, z_{cont} represents the continuous structure representation and z_{quant} represents a discrete structure representation of the protein. The loss function of the residual diffusion module 320 may be as follows:

$${ℒ}_{ϕ} = {𝔼}_{q ( r ) , ϵ ∼ 𝒩 ( 0 , I ) , t} [ \vert \vert ϵ - {ϵ}_{ϕ} ( {r}_{t} , t , h , {z}_{q u a n t} ) \vert {\vert}_{2}^{2} ] \qquad ( 4 )$$

where ϕ represents parameters of the residual diffusion module 320, t represents a time step, rₜ represents a residual obtained at the time step t.

After the residual r̅ is determined, a predicted continuous structure representation 325 (denoted as z̅_{cont}, and also referred to as continuous structure tokens) may be obtained based on the residual and the predicted discrete structure representation 310 (denoted as z̅_{zquant}). In an example, the residual may be added up to the predicted discrete structure representation 310 to recover the continuous structure tokens, which is closer to the features produced by the structure encoder 385. Then, the target structure 315 (e.g., an atomic structure) of the protein may be generated, by the structure decoder 390, based on the predicted continuous structure representation 325. In this way, the residual diffusion module 320 is capable of improving the structural prediction accuracy by refining fine-grained structural variations based on language model predictions. In addition, the residual diffusion module 320 performs fine-grained refinements on the local structure, optimizing interatomic distances to facilitate the formation of plausible secondary structures.

Although discrete structure tokenization efficiently captures high-level topology and enables co-generation, the transition to a latent-space language model inevitably sacrifices atomic-level details. However, this transition inherently disentangles geometric modeling from sequence-based generative modeling, with the structure tokenizer serving as an encoder-decoder in data space and the language model operating in the latent space of structure tokens. While this separation introduces information loss, it also presents an opportunity which indicates that the combination of the structure encoder, the language model, and the structure decoder as a whole effectively functions as a denoising model, capable of refining structure in atomic coordinates. In such a way, a structure denoising model may be defined as X_{θ}(xₜ, t):xₜ [symbol] x̅_{denoised} [symbol] decoder·PLM·encoder(xₜ). This structure denoising model (denoted as x_{θ}) may be seamlessly integrated into a generative framework. In addition, the structure denoising model may be incorporated into a flow-based sampler with Euler integrator, where each Euler step interpolates

$${x}_{s} ← \frac{s - t}{1 - t} \cdot {x}_{θ} ( {x}_{t} , t ) + \frac{1 - s}{1 - t} \cdot {x}_{t}$$

up to a Kabsch alignment of x̅_{denoised} against xₜ, treating it as a denoising process on data-space structure generation. The structure denoising model may be finetuned with flow matching (FM). This hybrid approach enables direct sampling in data space while preserving the scalability of discrete tokenization, ultimately improving atomic-level accuracy in protein modeling.

FIG. 3C illustrates a schematic diagram of an example process 300C of sampling with a flow-based sampler in accordance with some embodiments of the present disclosure. As shown in FIG. 3C, a transformation may be performed on a sample structure (also referred to as a denoised structure) 350 of the protein to obtain a transformed sample structure 355 of the protein. The transformation performed on the sample structure 350 may be an example of sampling with flow matching in a data space. The transformation may include a rotation and/or a translation performed on the sample structure 350. Then, the sample discrete structure representation may be obtained by encoding the transformed sample structure 355 of the protein using the structure encoder 385. Then, similar to the process described in FIG. 3A, at least one bit sequence in a sample discrete structure representation 360 is masked to obtain the masked discrete structure representation. Then, the language model 380 may generate the predicted discrete structure representation 365 at least based on the masked discrete structure representation. The structure decoder 390 may decode the predicted discrete structure representation 365 into the target structure 315. The language model 380 may be updated based on a difference between the between the target structure 315 and the sample structure 350. In this way, sampling with flow matching enhances the structure generation on the folding task, while supervision with the folding objective can bring further improvement.

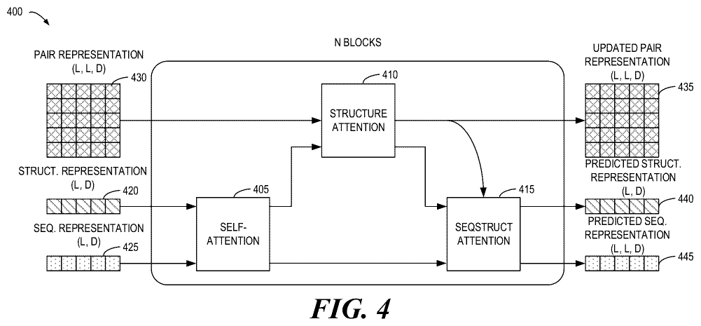

While bit-based modeling offers effective guide on the design of supervision targets, sequence-based models still lack the geometric inductive biases and structural learning objective that might be needed to capture the complexity of residual interactions. To address these limitations, geometric modules may be introduced with reference to FIG. 4, which illustrates a schematic diagram of a structure 400 of the language model 380 in accordance with some embodiments of the present disclosure. As shown in FIG. 4, the geometric modules include a self-attention module 405, a structure attention module 410 and a sequence structure attention module 415. The geometric modules may be integrated into encoder blocks of the language model 380, thereby operating on compact 2D pair representations to capture pairwise spatial dependencies of residues.

A first attention mechanism is applied, by the language model 380 (e.g., by the self-attention module 405), to a structure representation 420 of a protein and a sequence representation 425 of the protein, to obtain a first updated structure representation of the protein and a first updated sequence representation of the protein. A second attention mechanism is applied, by the language model 380 (e.g., the structure attention module 410), to a pair representation 430 of the protein and the first updated structure representation, to obtain an updated pair representation 435 of the protein and a second updated structure representation of the protein. The pair representation 430 may characterize interactions between pairs of amino acid residues in the protein. Then, a third attention mechanism is applied, by the language model 380 (e.g., the sequence structure attention module 415), to the updated pair representation 435, the second updated structure representation and the first updated sequence representation, to obtain at least one of a predicted structure representation 440 of the protein or a predicted sequence representation 445 of the protein. In this way, by combining the pair representations with sequence information and structure information, more structural patterns of protein may be capture, thereby improving structure prediction accuracy of the language model 380.

In some embodiments, in the training process of the language model 380, a portion of the structure representation 420 is masked, and a portion of the sequence representation 425 is masked. The language model 380 may be trained based on a difference between the structure representation 420 and the predicted structure representation 440 and/or a difference between the sequence representation 425 and the predicted sequence representation 445.

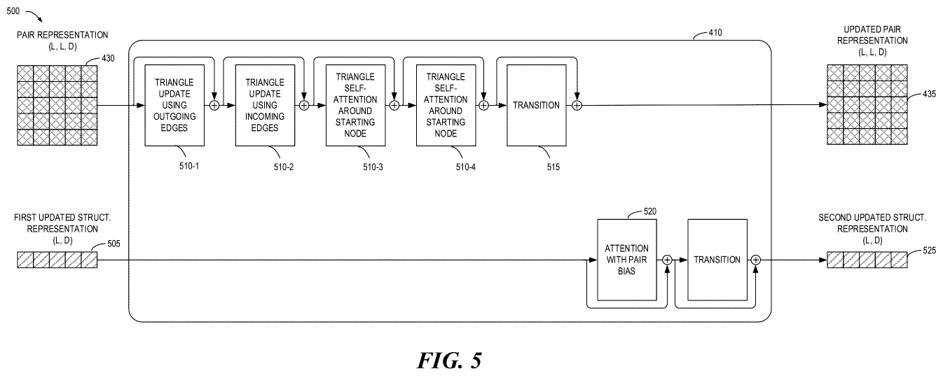

Details of the structure attention module 410 may be introduced with reference to FIG. 5, which illustrates a schematic diagram of a structure 500 of the structure attention module 410 in accordance with some embodiments of the present disclosure. As shown in FIG. 5, the input of the structure attention module 410 includes the pair representation 430 and the first updated structure representation 505 which is output by the self-attention module 405. The pair representation 430 may be denoted as (L, L, D) and the first updated structure representation 505 may be denoted as (L, D), where L denotes the number of residues and D denotes the feature dimension (e.g., 128 for the pair representation 430 and 1280 for the first updated structure representation 505). A triangle self-attention operation and a transition operation may be performed on the pair representation 430, to obtain the updated pair representation 435. For example, the structure attention module 410 performs the triangle self-attention operation on the pair representation 430 by a plurality of triangle operation blocks 510-1 to 510-4 and performs the transition operation on the pair representation 430 by a transition operation block 515. Then, an attention mechanism may be applied, at an attention block 520, to the first updated structure representation 505 by using the updated pair representation 435 as a bias, to obtain the second updated structure representation 525.

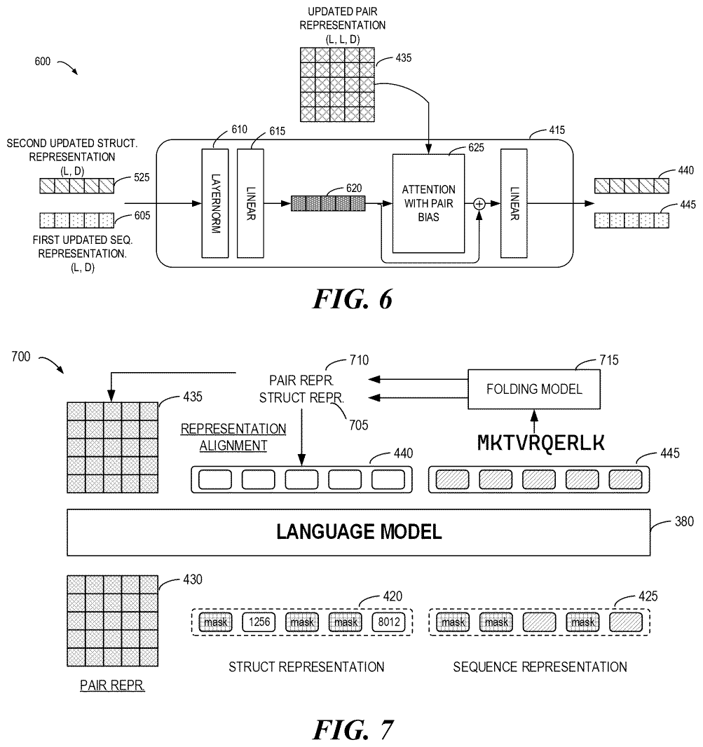

Details of the sequence structure attention module 415 may be introduced with reference to FIG. 6, which illustrates a schematic diagram of a structure 600 of the sequence structure attention module 415 in accordance with some embodiments of the present disclosure. As shown in FIG. 5, the input of the structure attention module 415 includes the updated pair representation 435, the second updated structure representation 525 and the first updated sequence representation 605. The second updated structure representation 525 and the first updated sequence representation 605 may be concatenated, e.g., at a layer normalization layer 610 and a linear layer 615, along a feature dimension, to obtain a concatenated representation 620. Then, an attention mechanism may be applied to the concatenated representation 620, at an attention block 625, by using the updated pair representation 435 as a bias, to obtain at least one of the predicted structure representation 440 or the predicted sequence representation 445.

Representations generated by the language model 380 may be aligned to representations from a folding model. FIG. 7 illustrates a schematic diagram of a process 700 of aligning the representations of the language model 380 to the representations from a folding model. As shown in FIG. 7, similar to the process in FIG. 4, the language model 380 may generate the updated pair representation 435, the predicted structure representation 440 and the predicted sequence representation 445 based on the pair representation 430, the structure representation 420 and the sequence representation 425. A reference structure representation 705 of the protein and a reference pair representation 710 of the protein may be generated, using a folding model 715, based on the predicted sequence representation 445. In an example, the folding model 715 may transform amino acid chains into specific 3D structures essential for protein function.

Then, a loss function may be determined based on a similarity (e.g., cosine similarity) between the reference pair representation 710 and the updated pair representation 435, and a similarity (e.g., cosine similarity) between the reference structure representation 705 and the predicted structure representation 440. In some examples, before aligning the updated pair representation 435 and the predicted structure representation 440 to the reference pair representation 710 and the reference structure representation 705, a 3-layer multilayer perceptron (MLP) may be used to project the updated pair representation 435 and the predicted structure representation 440 through negative cosine similarity. The language model 380 may be updated based on the loss function. In an example, the language model 380 may be updated based on a training objective, which is configured to reduce or minimize the value of the loss function. In this way, aligning the representations of the language model 380 to the representations of the folding model further diversifies generated structures, thereby improving structure prediction accuracy.

It is to be noted that a structure representation of a protein described with reference to FIG. 4 to FIG. 7 may be in any suitable format. For example, the structure representation may include the continuous structure representation, index-based discrete structure tokens, or bits-based discrete structure tokens. It is to be noted that embodiments described with reference to FIG. 3A to 3C and FIG. 7 may be implemented separately or implemented jointly.

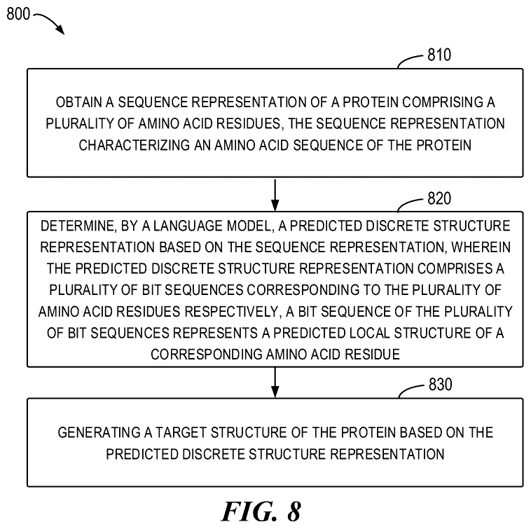

FIG. 8 illustrates a flowchart of a method 800 of protein structure generation in accordance with some example implementations of the present disclosure. The method 800 may be implemented at the electronic device 120 as illustrated in FIG. 1. At block 810, the electronic device 120 obtains a sequence representation of a protein comprising a plurality of amino acid residues, the sequence representation characterizing an amino acid sequence of the protein. At block 820, the electronic device 120 determines, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue. At block 830, the electronic device 120 generates a target structure of the protein based on the predicted discrete structure representation.

In some embodiments, generating the target structure of the protein comprises: determining a residual of the predicted discrete structure representation relative to a continuous structure representation of the protein based on a hidden state of the language model and the predicted discrete structure representation; obtaining a predicted continuous structure representation based on the residual and the predicted discrete structure representation; and generating, by a structure decoder, the target structure of the protein based on the predicted continuous structure representation.

In some embodiments, the method 800 is performed in training of the language model, the predicted discrete structure representation is determined further based on a masked discrete structure representation of the protein, and the method 800 further comprises: generating the masked discrete structure representation by masking at least one bit sequence in a sample discrete structure representation of the protein, a bit sequence in the sample discrete structure representation corresponds to an amino acid residue of the plurality of amino acid residues and represents a sample local structure of the corresponding amino acid residue; determining a loss function based on a difference between the predicted discrete structure representation and the sample discrete structure representation; and updating the language model based on the loss function.

In some embodiments, the method 800 further comprises performing a transformation on a sample structure of the protein to obtain a transformed sample structure of the protein, the transformation comprising at least one of a rotation or a translation; and obtaining the sample discrete structure representation by encoding the transformed sample protein, wherein the language model is updated based on a difference between the between the target structure and the sample structure.

In some embodiments, the protein comprises at least one of a multi-chain protein or a single-chain protein.

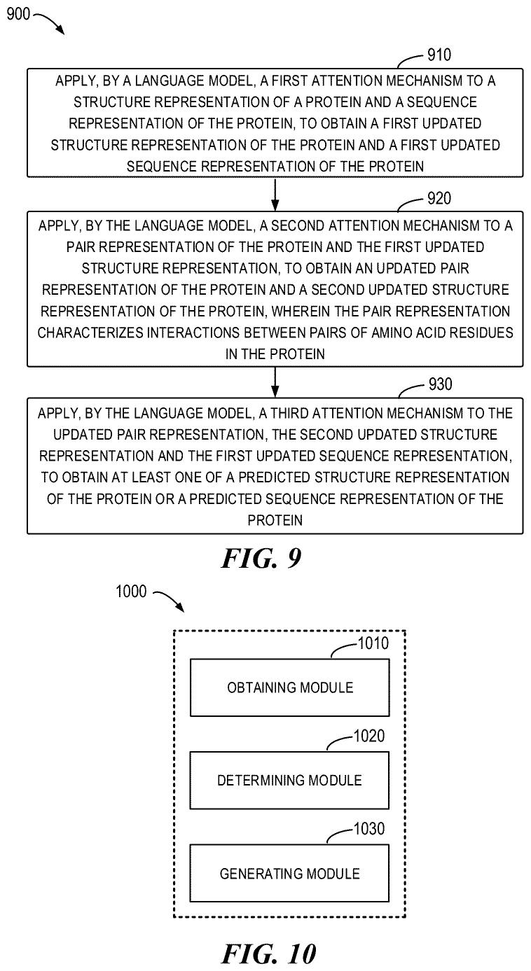

FIG. 9 illustrates a flowchart of a method 900 of protein structure generation in accordance with some example implementations of the present disclosure. The method 900 may be implemented at the electronic device 120 as illustrated in FIG. 1. At block 910, the electronic device 120 applies, by a language model, a first attention mechanism to a structure representation of a protein and a sequence representation of the protein, to obtain a first updated structure representation of the protein and a first updated sequence representation of the protein. At block 920, the electronic device 120 applies, by the language model, a second attention mechanism to a pair representation of the protein and the first updated structure representation, to obtain an updated pair representation of the protein and a second updated structure representation of the protein, wherein the pair representation characterizes interactions between pairs of amino acid residues in the protein. At block 930, the electronic device 120 applies, by the language model, a third attention mechanism to the updated pair representation, the second updated structure representation and the first updated sequence representation, to obtain at least one of a predicted structure representation of the protein or a predicted sequence representation of the protein.

In some embodiments, applying the second attention mechanism comprises: performing a triangle self-attention operation and a transition operation on the pair representation, to obtain the updated pair representation; and applying an attention mechanism to the first updated structure representation by using the updated pair representation as a bias, to obtain the second updated structure representation.

In some embodiments, applying the third attention mechanism comprises: concatenating the second updated structure representation and the first updated sequence representation along a feature dimension, to obtain a concatenated representation; and applying an attention mechanism to the concatenated representation by using the updated pair representation as a bias, to obtain at least one of the predicted structure representation or the predicted sequence representation.

In some embodiments, the method 900 is performed in training of the language model, a portion of the structure representation is masked, and a portion of the sequence representation is masked.

In some embodiments, the method 900 further comprises generating, using a folding model, a reference structure representation of the protein and a reference pair representation of the protein based on the predicted sequence representation; determining a loss function based on a similarity between the reference pair representation and the updated pair representation, and a similarity between the reference structure representation and the predicted structure representation; and updating the language model based on the loss function.

FIG. 10 shows a block diagram of an apparatus 1000 for protein structure generation in accordance with some embodiments of the present disclosure. The apparatus 1000 may be implemented, for example, or included at the electronic device 120 of FIG. 1. Various modules/components in the apparatus 1000 may be implemented by hardware, software, firmware, or any combination thereof.

As illustrated, the apparatus 1000 includes an obtaining module 1010 configured to obtain a sequence representation of a protein comprising a plurality of amino acid residues, the sequence representation characterizing an amino acid sequence of the protein; a determining module 1020 configured to determine, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue; and a generating module 1030 configured to generate a target structure of the protein based on the predicted discrete structure representation.

In some embodiments, the generating module 1030 is further configured to determine a residual of the predicted discrete structure representation relative to a continuous structure representation of the protein based on a hidden state of the language model and the predicted discrete structure representation; obtain a predicted continuous structure representation based on the residual and the predicted discrete structure representation; and generate, by a structure decoder, the target structure of the protein based on the predicted continuous structure representation.

In some embodiments, the apparatus 1000 may further comprise a training module and the predicted discrete structure representation may be determined further based on a masked discrete structure representation of the protein. The training module may be configured to generate the masked discrete structure representation by masking at least one bit sequence in a sample discrete structure representation of the protein, a bit sequence in the sample discrete structure representation corresponds to an amino acid residue of the plurality of amino acid residues and represents a sample local structure of the corresponding amino acid residue; determine a loss function based on a difference between the predicted discrete structure representation and the sample discrete structure representation; and update the language model based on the loss function.

In some embodiments, the training module may be configured to perform a transformation on a sample structure of the protein to obtain a transformed sample structure of the protein, the transformation comprising at least one of a rotation or a translation; and obtain the sample discrete structure representation by encoding the transformed sample protein, wherein the language model is updated based on a difference between the between the target structure and the sample structure.

In some embodiments, the protein comprises at least one of a multi-chain protein or a single-chain protein.

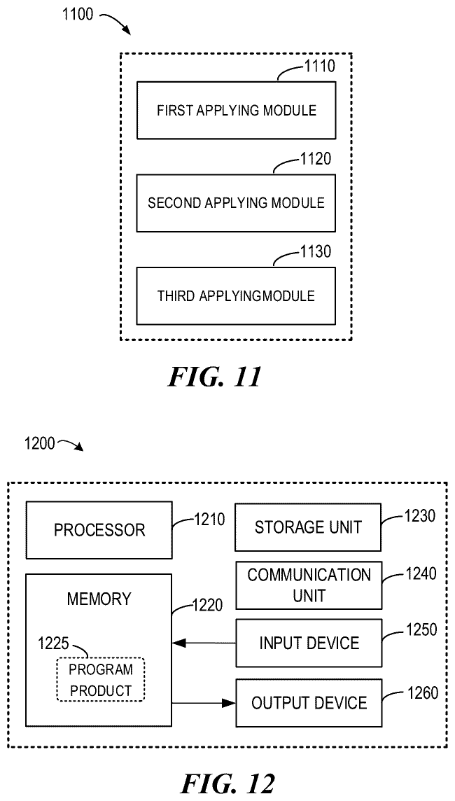

FIG. 11 shows a block diagram of an apparatus 1100 for protein structure generation in accordance with some embodiments of the present disclosure. The apparatus 1100 may be implemented, for example, or included at the electronic device 120 of FIG. 1. Various modules/components in the apparatus 1100 may be implemented by hardware, software, firmware, or any combination thereof.

As illustrated, the apparatus 1000 includes a first applying module 1110 configured to apply, by a language model, a first attention mechanism to a structure representation of a protein and a sequence representation of the protein, to obtain a first updated structure representation of the protein and a first updated sequence representation of the protein; a second applying module 1120 configured to apply, by the language model, a second attention mechanism to a pair representation of the protein and the first updated structure representation, to obtain an updated pair representation of the protein and a second updated structure representation of the protein, wherein the pair representation characterizes interactions between pairs of amino acid residues in the protein; and a third applying module 1130 configured to apply, by the language model, a third attention mechanism to the updated pair representation, the second updated structure representation and the first updated sequence representation, to obtain at least one of a predicted structure representation of the protein or a predicted sequence representation of the protein.

In some embodiments, the second applying module 1120 may be further configured to perform a triangle self-attention operation and a transition operation on the pair representation, to obtain the updated pair representation; and apply an attention mechanism to the first updated structure representation by using the updated pair representation as a bias, to obtain the second updated structure representation.

In some embodiments, the third applying module 1130 may be further configured to concatenating the second updated structure representation and the first updated sequence representation along a feature dimension, to obtain a concatenated representation; and applying an attention mechanism to the concatenated representation by using the updated pair representation as a bias, to obtain at least one of the predicted structure representation or the predicted sequence representation.

In some embodiments, the apparatus 1000 may comprise a training module configured to train the language model. A portion of the structure representation is masked, and a portion of the sequence representation is masked.

In some embodiments, the training module may be configured to generate, using a folding model, a reference structure representation of the protein and a reference pair representation of the protein based on the predicted sequence representation; determine a loss function based on a similarity between the reference pair representation and the updated pair representation, and a similarity between the reference structure representation and the predicted structure representation; and update the language model based on the loss function.

FIG. 12 illustrates a block diagram of an electronic device 1200 in which one or more embodiments of the present disclosure can be implemented. It would be appreciated that the electronic device 1200 shown in FIG. 12 is only an example and should not constitute any restriction on the function and scope of the embodiments described herein. The electronic device 1200 may be used, for example, to implement the electronic device 120 of FIG. 1. The electronic device 1200 may also be used to implement the apparatus 1000 of FIG. 10 or the apparatus 1100 of FIG. 11.

As shown in FIG. 12, the electronic device 1200 is in the form of a general computing device. The components of the electronic device 1200 may include, but are not limited to, one or more processing units or processors 1210, a memory 1220, a storage device 1230, one or more communication units 1240, one or more input devices 1250, and one or more output devices 1260. The processor 1210 may be an actual or virtual processor and can execute various processes according to the programs stored in the memory 1220. In a multiprocessor system, multiple processors execute computer executable instructions in parallel to improve the parallel processing capability of the electronic device 1200.

The electronic device 1200 typically includes a variety of computer storage medium. Such medium may be any available medium that is accessible to the electronic device 1200, including but not limited to volatile and non-volatile medium, removable and non-removable medium. The memory 1220 may be volatile memory (for example, a register, cache, a random access memory (RAM)), a non-volatile memory (for example, a read-only memory (ROM), an electrically erasable programmable read-only memory (EEPROM), a flash memory) or any combination thereof. The storage device 1230 may be any removable or non-removable medium, and may include a machine-readable medium, such as a flash drive, a disk, or any other medium, which can be used to store information and/or data (such as training data for training) and can be accessed within the electronic device 500.

The electronic device 1200 may further include additional removable/non-removable, volatile/non-volatile, transitory/non-transitory storage medium. Although not shown in FIG. 12, a disk driver for reading from or writing to a removable, non-volatile disk (such as a “floppy disk”), and an optical disk driver for reading from or writing to a removable, non-volatile optical disk can be provided. In these cases, each driver may be connected to the bus (not shown) by one or more data medium interfaces. The memory 1220 may include a computer program product 1225, which has one or more program modules configured to perform various methods or acts of various embodiments of the present disclosure.

The communication unit 1240 communicates with a further computing device through the communication medium. In addition, functions of components in the electronic device 1200 may be implemented by a single computing cluster or multiple computing machines, which can communicate through a communication connection. Therefore, the electronic device 1200 may be operated in a networking environment using a logical connection with one or more other servers, a network personal computer (PC), or another network node.

The input device 1250 may be one or more input devices, such as a mouse, a keyboard, a trackball, etc. The output device 1260 may be one or more output devices, such as a display, a speaker, a printer, etc. The electronic device 1200 may also communicate with one or more external devices (not shown) through the communication unit 1240 as required. The external device, such as a storage device, a display device, etc., communicate with one or more devices that enable users to interact with the electronic device 1200, or communicate with any device (for example, a network card, a modem, etc.) that makes the electronic device 1200 communicate with one or more other computing devices. Such communication may be executed via an input/output (I/O) interface (not shown).

According to example implementation of the present disclosure, a computer-readable storage medium is provided, on which a computer-executable instruction or computer program is stored, where the computer-executable instructions or the computer program is executed by the processor to implement the method described above. According to example implementation of the present disclosure, a computer program product is also provided. The computer program product is physically stored on a non-transient computer-readable medium and includes computer-executable instructions, which are executed by the processor to implement the method described above.

Various aspects of the present disclosure are described herein with reference to the flow chart and/or the block diagram of the method, the device, the equipment and the computer program product implemented in accordance with the present disclosure. It would be appreciated that each block of the flowchart and/or the block diagram and the combination of each block in the flowchart and/or the block diagram may be implemented by computer-readable program instructions.

These computer-readable program instructions may be provided to the processors of general-purpose computers, special computers or other programmable data processing devices to produce a machine that generates a device to implement the functions/acts specified in one or more blocks in the flow chart and/or the block diagram when these instructions are executed through the processors of the computer or other programmable data processing devices. These computer-readable program instructions may also be stored in a computer-readable storage medium. These instructions enable a computer, a programmable data processing device and/or other devices to work in a specific way. Therefore, the computer-readable medium containing the instructions includes a product, which includes instructions to implement various aspects of the functions/acts specified in one or more blocks in the flowchart and/or the block diagram.

The computer-readable program instructions may be loaded onto a computer, other programmable data processing apparatus, or other devices, so that a series of operational steps can be performed on a computer, other programmable data processing apparatus, or other devices, to generate a computer-implemented process, such that the instructions which execute on a computer, other programmable data processing apparatus, or other devices implement the functions/acts specified in one or more blocks in the flowchart and/or the block diagram.

The flowchart and the block diagram in the drawings show the possible architecture, functions and operations of the system, the method and the computer program product implemented in accordance with the present disclosure. In this regard, each block in the flowchart or the block diagram may represent a part of a module, a program segment or instructions, which contains one or more executable instructions for implementing the specified logic function. In some alternative implementations, the functions marked in the block may also occur in a different order from those marked in the drawings. For example, two consecutive blocks may actually be executed in parallel, and sometimes can also be executed in a reverse order, depending on the function involved. It should also be noted that each block in the block diagram and/or the flowchart, and combinations of blocks in the block diagram and/or the flowchart, may be implemented by a dedicated hardware-based system that performs the specified functions or acts, or by the combination of dedicated hardware and computer instructions.

Each implementation of the present disclosure has been described above. The above description is example, not exhaustive, and is not limited to the disclosed implementations. Without departing from the scope and spirit of the described implementations, many modifications and changes are obvious to ordinary skill in the art. The selection of terms used in this article aims to best explain the principles, practical application or improvement of technology in the market of each implementation, or to enable other ordinary skill in the art to understand the various embodiments disclosed herein.

1. A method of protein structure generation, comprising:
obtaining a sequence representation of a protein comprising a plurality of amino acid residues, the sequence representation characterizing an amino acid sequence of the protein;
determining, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue; and
generating a target structure of the protein based on the predicted discrete structure representation.

2. The method of claim 1, wherein generating the target structure of the protein comprises:
determining a residual of the predicted discrete structure representation relative to a continuous structure representation of the protein based on a hidden state of the language model and the predicted discrete structure representation;
obtaining a predicted continuous structure representation based on the residual and the predicted discrete structure representation; and
generating, by a structure decoder, the target structure of the protein based on the predicted continuous structure representation.

3. The method of claim 1, wherein the method is performed in training of the language model, the predicted discrete structure representation is determined further based on a masked discrete structure representation of the protein, and the method further comprises:
generating the masked discrete structure representation by masking at least one bit sequence in a sample discrete structure representation of the protein, a bit sequence in the sample discrete structure representation corresponds to an amino acid residue of the plurality of amino acid residues and represents a sample local structure of the corresponding amino acid residue;
determining a loss function based on a difference between the predicted discrete structure representation and the sample discrete structure representation; and
updating the language model based on the loss function.

4. The method of claim 3, further comprising:
performing a transformation on a sample structure of the protein to obtain a transformed sample structure of the protein, the transformation comprising at least one of a rotation or a translation; and
obtaining the sample discrete structure representation by encoding the transformed sample protein, wherein the language model is updated based on a difference between the between the target structure and the sample structure.

5. The method of claim 3, wherein the protein comprises at least one of a multi-chain protein or a single-chain protein.

6. A method of protein structure generation, comprising:
applying, by a language model, a first attention mechanism to a structure representation of a protein and a sequence representation of the protein, to obtain a first updated structure representation of the protein and a first updated sequence representation of the protein;
applying, by the language model, a second attention mechanism to a pair representation of the protein and the first updated structure representation, to obtain an updated pair representation of the protein and a second updated structure representation of the protein, wherein the pair representation characterizes interactions between pairs of amino acid residues in the protein;
applying, by the language model, a third attention mechanism to the updated pair representation, the second updated structure representation and the first updated sequence representation, to obtain at least one of a predicted structure representation of the protein or a predicted sequence representation of the protein.

7. The method of claim 6, wherein applying the second attention mechanism comprises:
performing a triangle self-attention operation and a transition operation on the pair representation, to obtain the updated pair representation; and
applying an attention mechanism to the first updated structure representation by using the updated pair representation as a bias, to obtain the second updated structure representation.

8. The method of claim 6, wherein applying the third attention mechanism comprises:
concatenating the second updated structure representation and the first updated sequence representation along a feature dimension, to obtain a concatenated representation; and
applying an attention mechanism to the concatenated representation by using the updated pair representation as a bias, to obtain at least one of the predicted structure representation or the predicted sequence representation.

9. The method of claim 6, wherein the method is performed in training of the language model, a portion of the structure representation is masked, and a portion of the sequence representation is masked.

10. The method of claim 9, further comprising:
generating, using a folding model, a reference structure representation of the protein and a reference pair representation of the protein based on the predicted sequence representation;
determining a loss function based on a similarity between the reference pair representation and the updated pair representation, and a similarity between the reference structure representation and the predicted structure representation; and
updating the language model based on the loss function.

11. An electronic device, comprising:
at least one processor; and
at least one memory coupled to the at least one processor and storing instructions executable by the at least one processor, the instructions, upon execution by the at least one processor, causing the electronic device to perform operations comprising:
obtaining a sequence representation of a protein comprising a plurality of amino acid residues, the sequence representation characterizing an amino acid sequence of the protein;
determining, by a language model, a predicted discrete structure representation based on the sequence representation, wherein the predicted discrete structure representation comprises a plurality of bit sequences corresponding to the plurality of amino acid residues respectively, a bit sequence of the plurality of bit sequences represents a predicted local structure of a corresponding amino acid residue; and
generating a target structure of the protein based on the predicted discrete structure representation.

12. The electronic device of claim 11, wherein generating the target structure of the protein comprises:
determining a residual of the predicted discrete structure representation relative to a continuous structure representation of the protein based on a hidden state of the language model and the predicted discrete structure representation;
obtaining a predicted continuous structure representation based on the residual and the predicted discrete structure representation; and
generating, by a structure decoder, the target structure of the protein based on the predicted continuous structure representation.

13. The electronic device of claim 11, wherein the operations are performed in training of the language model, the predicted discrete structure representation is determined further based on a masked discrete structure representation of the protein, and the operations further comprises:
generating the masked discrete structure representation by masking at least one bit sequence in a sample discrete structure representation of the protein, a bit sequence in the sample discrete structure representation corresponds to an amino acid residue of the plurality of amino acid residues and represents a sample local structure of the corresponding amino acid residue;
determining a loss function based on a difference between the predicted discrete structure representation and the sample discrete structure representation; and
updating the language model based on the loss function.

14. The electronic device of claim 13, the operations further comprising:
performing a transformation on a sample structure of the protein to obtain a transformed sample structure of the protein, the transformation comprising at least one of a rotation or a translation; and
obtaining the sample discrete structure representation by encoding the transformed sample protein, wherein the language model is updated based on a difference between the between the target structure and the sample structure.

15. The electronic device of claim 13, wherein the protein comprises at least one of a multi-chain protein or a single-chain protein.
